(19) United States
(12) Patent Application Publication
(10) Pub. No.: US 2011/0124949 A1
(43) Pub. Date: May 26, 2011

(54) METHOD AND APPARATUS FOR STABILIZING TUBING DURING A BRACHYTHERAPY PROCEDURE

(75) Inventor: Cynthia Ruth Henke-Sarmento, Lakewood, CO (US)

(73) Assignee: QSUM Biopsy Disposables LLC, Boulder, CO (US)

(21) Appl. No.: 12/625,937

(22) Filed: Nov. 25, 2009

(51) Int. Cl. A61M 36/12

(52) U.S. Cl. 600/7

(57) ABSTRACT

A stabilization device that may be used in connection with a radiation therapy procedure such as a breast brachytherapy procedure to stabilize one or more tubular members of a brachytherapy device between treatment sessions. The device includes a base member for placement over a body site of a patient and a cover member that may be positioned over the base member. The one or more tubular members may be locatable between the base and cover members and thereby stabilized relative to at least one of the base and cover members. In some embodiments, the base member may be in the form of a sheet member, and the stabilization device may include one or more resilient members that may be placed under the tubular members to limit lateral movement of the tubular members and provide a cushioning effect for the patient.

### FIELD OF THE INVENTION

This invention relates generally to the field of brachytherapy and more specifically to a process and apparatus for stabilizing tubing used as part of a breast brachytherapy procedure.

### BACKGROUND OF THE INVENTION

Brachytherapy (e.g., sealed source radiotherapy, endocurietherapy, temporary radiation implant) is a form of radiotherapy involving placing a radioactive source inside or next to an area requiring treatment (e.g., tumor area), and is commonly used to treat cancers such as prostate cancer, cervical cancer, and cancers of the head and neck. This technique delivers a concentrated dose of radiation to a restricted volume while minimizing radiation effects on normal tissue. Traditionally, the concentrated dose of radiation has been administered in the form of radioactive seeds (e.g., radioactive Iodine (I-125) or palladium (Pd-103)) that a doctor implants into a tumor area (e.g., prostate gland).

More recently, brachytherapy has been used in the treatment of breast cancer after removal of one or more tumors from the breast (e.g., lumpectomy). One type of breast brachytherapy involves inserting a number of plastic catheters into the affected breast through individual incisions in the breast such that the ends of the catheters are in or near a cavity where the tumor previously existed. Typically, the patient is lying on his or her back during the procedure. Thereafter, twice a day for the next 5-7 days (“treatment period”), the catheters are briefly connected to a radiation source to administer radiation to portions of cavity during a “treatment session”. The catheters remain in the breast during the entire treatment period.

Another type of breast brachytherapy involves utilization of a brachytherapy device that generally includes a bundle of catheters or tubes with lumens therein. In one arrangement, the bundle of catheters is inserted into the cavity and a balloon surrounding the catheters is inflated to maintain the shape of the cavity and/or reduce contact between the catheters in the cavity and the tissue surrounding the cavity. In another arrangement, the portions of the catheters residing within the cavity are appropriately expanded to resemble a “wire whip” or other similar device. In either case, a radiation source is delivered into one or more of the catheters twice a day during the treatment period to treat the affected areas. The catheters and balloon remain in the breast during the entire treatment period.

The radiation source may remain within the catheters or balloon during the entire treatment period, and in this regard, may be in the form of radioactive seeds or other sources. Alternatively, the radiation source may be in the form of an X-ray source that that may be inserted into the catheter and eventually into the balloon and/or cavity. Thereafter, the X-ray source (e.g., camera) may remain in the balloon and deliver radiation for any appropriate amount of time (e.g., 10 minutes). The X-ray source may then be turned off and withdrawn through the catheter, and thus no radiation may remain in the breast between treatments or after the final treatment.

Brachytherapy may be advantageous over other available methods and manners of treating breast cancer. For instance, brachytherapy may expose patients to substantially less radiation than does traditional external beam radiation and consumes substantially less time than does the external beam radiation (e.g., 5-7 days versus 6-7 weeks). It may also be advantageous over a mastectomy as the patient retains the affected breast at the conclusion of the procedure.

### SUMMARY OF THE INVENTION

In view of the foregoing, one objective of the present invention is to stabilize (e.g., reduce movement of) brachytherapy devices that have been percutaneously positioned at a body site of a patient after a surgical procedure such as a lumpectomy.

Another objective of the invention is to reduce the volume of imaging procedures that may be necessary to confirm placement of the brachytherapy devices in a target area or cavity of the patient.

Another objective is to increase patient comfort between therapy sessions during a brachytherapy treatment period.

Another objective is to provide an apparatus that addresses one or more of the foregoing objectives in a manner that is easy to employ and/or otherwise implement at a low cost.

In addressing one or more of the above objectives and in accordance with one embodiment of the invention, there is disclosed an apparatus for use in connection with a radiation therapy procedure (e.g., brachytherapy) in which a radioactive substance is administered through at least one tube that is percutaneously positionable at a body site. The apparatus includes a base member that is positionable over a portion of the body site and a cover member that is positionable over the base member.

According to one aspect, the cover member may be one of interconnected and interconnectable to the base member and at least a portion of the at least one tube (e.g., a portion protruding from and extending away from the body site) may be locatable between the cover member and the base member to stabilize the portion of the at least one tube in a desired position. For instance, one desired position may be relative to at least one of the base and cover members.

The base member may generally be any flexible layer, member or barrier of any appropriate dimensions and shape (e.g., rectangular, circular) that is operable to be positioned over a body site of a patient (e.g., a breast or other location where a radiation therapy procedure is to be performed). In one embodiment, the base member may be in the form of a sheet member constructed of a hydrophobic, breathable material. In this regard, the sheet member may be resistant to the passage of liquids from one side to another side while allowing for the passage of gases (e.g., air). This construction may increase patient comfort by reducing sweating for some patients between the sheet member and the body site. Other materials (e.g., resilient materials such as foam) and constructions area also contemplated for the sheet member. In some embodiments, one or more of the sheet and cover members may be constructed of non-latex materials so as to reduce the risk of allergic reactions.

The cover member may also be in the form of any flexible layer or member of any appropriate dimensions and shape that may be positioned over the base member and may, together with the base member, stabilize one or more tubular members (hereinafter “tubular members,” even though it should be understood that tubular members could also encompass only a single tube or tubular member) in a desired position. For instance, the cover member may be of a shape and dimensions that are the same as or substantially similar to those of the base member. In other variations, the cover member may be operable to cover the tubular members. In one embodiment, the cover member may be absorbent and in this regard may be operable to absorb or otherwise wick up fluids associated with the brachytherapy procedure and/or device(s) (e.g., sweat, blood, radioactive substances) and protect the patient and personnel from such fluids. For example, the cover member may include an absorbent layer with a non-absorbent sheath or layer surrounding at least a portion of the absorbent layer. In this regard, the portion of the absorbent layer not covered by the sheath may be limited to the portion of the cover member in contact with the tubular members (e.g., catheter exit wires). As such, a top portion of the cover member may include the sheath and may allow the patient or technician to contact the top of the cover member without contacting any liquids retained by the absorbent layer. Additionally, this construction may provide protection for the patient's clothing (e.g., bra, shirt). In other embodiments, the cover member may be constructed of one or more resilient materials (e.g., foam).

In one embodiment, the cover member and base member may be pivotally connected along an adjoinment region. Pivotally connecting the cover and base members together may allow an operator to efficiently position the cover member over the sheet member by simply flapping or pivoting the cover member over the base member. This feature also may simplify mounting of the apparatus over the body site. In one arrangement, the cover member may be pivotal between at least opened and closed positions relative to the base member. In another arrangement, the adjoinment region may extend along a first edge of each of the cover member and base member. For instance, the base member may be in the form of a sheet member and the apparatus may further include an opening positioned adjacent to a second edge of the sheet member for receiving the tubular members therethrough to locate the apparatus relative to the body site. Here, the first edge of the sheet member may be located opposite the second edge of the sheet member. Stated otherwise, the opening that may receive the tubular members may be located on an opposite edge of the sheet member than is the edge that the adjoinment region extends along. This feature may advantageously allow the cover and sheet members to be effective contain and stabilize the ends of the tubular members. In another arrangement, the tubular members may each include a free end operable to be received between the cover member and the sheet member near the adjoinment region. This positioning of the tubular members may act to limit upward and downward as well as lateral movement of the portions of the tubular members as the distance between an upper surface of the sheet member and a lower surface of the cover member eventually may go to zero near the adjoinment region.

In another embodiment, the apparatus may include any appropriate anchoring element operable to locate the apparatus adjacent the body site. For instance, the anchoring element may include an opening extending through the base member that is sized for receiving the tubular members therethrough so as to locate the apparatus adjacent the body site. In this regard, location or extension of the tubular members through the opening may serve to removably secure the apparatus relative to the body site between therapy sessions. Once the apparatus is removed from the body site, a new apparatus may be removably secured relative to the body site after later therapy sessions.

In one arrangement, the opening may be at least partially defined by at least one hole (e.g., circular, elliptical, elongated, square). For instance, the hole may be located adjacent an edge of the sheet member and as a result, the tubular members may be efficiently inserted into or otherwise received within the hole. In another arrangement, the opening may additionally or alternatively be defined by at least one slit. For instance, the slit may extend away from the at least one hole to provide access to the hole for the tubular members and may be of a width less than a diameter of the hole. In one variation, the at least one slit may extend to an edge of the base member. As such, the tubular members may be located through the slit and into the hole whereby the tubular members may be removably secured within the opening until the patient or technician urges the apparatus away from the tubular members such that the tubular members travel back through the slit to a location separate from the apparatus.

In some embodiments disclosed herein, the apparatus may further include at least one orientation member operable to orient the portion of the tubular members relative to at least one of the base member (e.g., sheet member) and the cover member. For instance, the orientation member may orient the portions relative to a receiving area on a sheet member. The receiving area may extend along a length of the sheet member or any other portion where it is desired that the tubular members extend along. In one arrangement, the orientation member may be in the form of a resilient member that is positionable between the portions of the tubular members and the sheet member, and the portions may be locatable over the resilient member. In this regard, the resilient member may serve numerous purposes such as stabilizing the portions of the tubular members or otherwise further limiting movement of the portions once the cover member is positioned over the portions and the sheet member, relieving strain, kinking, bending, and/or rotation of the tubular members as the tubular members exit and protrude away from the body side, providing a padding or cushioning effect for the patient that limits the degree to which the tubular members can dig or burrow into a patient's skin, and the like.

The resilient member may be formed of any appropriate lightweight material that may serve one or more of the above purposes. For instance, the resilient member may be constructed of foam (e.g., flexible polyurethane, sponge rubber) and may be in any appropriate shape (e.g., block-shaped, layered) and of any appropriate dimensions. In one variation, the resilient member may include a channel on a surface thereof that is operable to receive the portion of the at least one tube. For instance, the channel may extend along a length of the resilient member and may be sized to receive the tubular members. In this regard, it should be appreciated that the channel may serve to limit lateral (e.g., side-to-side) movement of the tubular members and assist in locating or aligning the tubular members with a desired portion of the sheet and or cover members. For instance, the resilient member may be separable from the sheet and cover member and in this regard, a user may position the resilient member at a desired location and then position the portions of the tubular members within the channel.

In another variation, the resilient member may be configured in a wedge shape. For instance, the resilient member may include a first end having a first height and an opposite second end having a second height that is greater than the first height. In one arrangement, the first end may be positionable towards the body site and the second end may be positionable away from the body site which may serve to provide a more gradual exit angle for the portions of the tubular members as the portions exit the body site to thereby reduce kinking, bending and/or rotation of the tubular members.

While use of a single resilient member has been discussed, it may be desirable in some situations to utilize multiple resilient members to obtain a desired orientation or positioning of the tubular members and/or enhance patient comfort. For instance, some brachytherapy devices include a rigid (e.g., non-bendable) tubular member that exits and protrudes from the body site for a particular distance (e.g., 3-4 cm) in addition to a number of flexible tubular members (e.g., catheters) protruding from the rigid tubular member. In this situation, it may be desirable to stack a plurality of resilient members to a height that is at least approximately equal to a length or height of the rigid tubular member to provide support or stabilization that limits the degree to which the rigid tubular member can fall or tip over while providing a support surface for the flexible tubular members. Other arrangements and placements of multiple resilient members are also envisioned such as side by side positioning and the like.

In another arrangement, the orientation member may include a first securing element that is one of interconnected or interconnectable to at least one of the sheet member and the cover member and that is operable to selectively removably secure the cover member to the sheet member. In a variation, the orientation member may also include a second securing element that is one of interconnected or interconnectable to at least one of the sheet member and the cover member and that is operable to selectively removably secure the cover member to the sheet member. One or more of the first and second securing elements may include any appropriate adhesive surface or layer (e.g., sticky tape) with a backing sheet removably adhered thereto to selectively expose the adhesive surface or layer. As another example, corresponding hook and loop elements may be interconnected or interconnectable to the sheet and cover members to removably secure the cover and sheet members together. Other types of securing elements are also contemplated such as hook and loop, rubber bands, string, tape and the like.

It will be appreciated that the first and second securing elements may be interconnected or interconnectable to an appropriate portions of the sheet and/or cover members. For instance, the first and second securing elements may be arranged on opposite sides of a longitudinal axis of one of the sheet member and the cover member. In this arrangement, the portions of the tubular members and may be positioned over the sheet member and between the first and second securing element. In this regard, after the cover member is brought or otherwise positioned over the sheet member and the portions of the tubular members and removably secured to the sheet member via the first and second securing elements, the first and second securing elements may serve to limit lateral movement of the portions of the tubular members in addition to indirectly limiting upward and downward movement of the portions of the tubular members.

In another variation, the securing elements may be used in conjunction with the resilient element(s) to stabilize the portions of the tubular members. For instance, the resilient member may be sized for positioning between the first and second securing elements such that the portions of the tubular members maybe positioned thereover. As previously discussed, the resilient member may include a channel in a surface thereof (e.g., a top surface) which may receive the portions of the tubular members, and this channel may also extend between the first and second securing elements and in one specific setup, may extend along and be alignable over a longitudinal axis of the sheet member.

In another embodiment, the apparatus may include one or more absorbent members positionable between the body site and the sheet member. For instance, each absorbent member may be a drain sponge or other absorbent layer that may be sterile and operable to wick up or absorb liquids associated with the brachytherapy procedure. In other embodiments and in the situation where the portion of the apparatus contacting the patient is already sterile (e.g., the sheet member), the one or more absorbent layers may not be necessary.

In another aspect an apparatus is provided for use in connection with a radiation therapy procedure in which a radioactive substance is administered through at least one tube that is percutaneously positionable at a body site. The apparatus includes a sheet member that is positionable over a portion of the body site, an opening extending through the sheet member for receiving the at least one tube therethrough to locate the apparatus relative to the body site, and a cover member positionable over the sheet member. Here, a portion of the at least one tube may be locatable between the cover member and the sheet member and is thereby stabilized in a desired position.

According to another arrangement or embodiment, each of the base and cover members may be in the form of first and second resilient members (e.g., foam and the like) that may be used to “sandwich” and/or compress around the portions of the tubular members. For instance, each of the first and second resilient members may resemble the above discussed “resilient member”. In one variation, separate first and second resilient members may be positioned above and below the tubular member portions and may thereafter be removably secured together via any of the above discussed securing element(s). For instance, a technician may place a rubber band around the first and second resilient members to removably secure the first and second resilient members together and thus stabilize the portions of the tubular members between the first and second resilient members. In another variation, a single resilient member that has been sliced or otherwise cut partially therethrough may form the first and second resilient members and thus resemble a “clamshell” type member. In this regard, a technician may insert or position the tubular member portions in between the first and second resilient members or clamshells (e.g., like a hotdog in a bun) and then appropriately removably secure the clamshells together.

In another variation, one or more of the first and second resilient members may include one or more orientation members or elements operable to orient or stabilize the tubular members relative to at least one of the first and second resilient members. For instance, one or more of the resilient members may include a channel on a surface thereof for receipt of the tubular members to thereby stabilize the tubular members against lateral movement. In one approach, both of the first and second resilient members may include at least one channel that overly each other that collectively operate to contain the tubular members. In another approach, one or more of the first and second resilient members may include multiple channels each of which operations to accommodate one or more tubular members. In a further variation, one or more of the resilient members may include any of the above discussed anchoring elements (e.g., opening, cutout, hook and loop straps) operable to at least temporarily anchor or secure the first and/or second resilient member to the tubular members.

Some embodiments of the present invention provide various methodologies for use in connection with a radiation therapy procedure in which a radioactive substance is administered through at least one tube that is percutaneously positionable at a body site. Any of the methodologies may include initially appropriately cleaning and/or sterilizing the body with any appropriate substances or solutions. In a broad aspect, one method may include locating at least a portion of the at least one tube over a base member and positioning a cover member over at least a portion of the base member and the portion of the at least one tube. The portion of the at least one tube may be located between the cover member and the base member to stabilize the portion of the at least one tube (hereinafter “tubular members,” even though it should be understood that tubular members could also encompass only a single tube or tubular member) in a desired position.

In one embodiment, the base member may be in the form of a sheet member and the locating step may include routing the portions of the tubular members through an opening in the sheet member. For instance, the portions may be disposed along a slit or slot and into a receiving hole in the sheet member to removably anchor at least the sheet member to the tubular members. In another embodiment, the locating step may include disposing the portions of the tubular members along a length of the base member. For example, some arrangements envision that sheet member may accommodate up to a 9 inch length of tubular members (e.g., catheter wires).

In another embodiment, the method may include orienting the portions of the tubular members relative to at least one of the base member and the cover member. This step may include limiting movement of the portions of the tubular members relative to the base and/or cover members and/or limiting access to the portions of the tubular members. In one variation, the base member may be in the form of a sheet member and the orienting step may further include disposing a resilient member (e.g., flexible block or wedge-shaped member) between the portions of the tubular members and the sheet member. The disposing step may be subject to a number of characterizations. In one arrangement, it may include placing the resilient member along a longitudinal axis of the sheet member. This arrangement of the resilient member may advantageously allow the resilient member to accept or receive the portions of the tubular members as the portions exit the body site onto the sheet member. In another arrangement, the resilient member may include a first end having a first height and a second end having a second height that is greater than the first height. Here, the disposing step may include arranging the first end to be near the body site and arranging the second end to be away from the body site. This arrangement may reduce kinking, bending and/or rotating of the tubular members. A further arrangement envisions that the portions may be aligned with a channel in a surface of the resilient member. As also discussed above, locating the portions of the tubular members in the channel may reduce or limit at least lateral movement of the portions.

In another variation, the orienting step may include removably securing the cover member to the base member using at least one securing element (e.g., adhesive strip, hook and loop piece(s), rubber band) that is connected to at least one of the cover member and the sheet member. For instance, the at least one securing element may include first and second securing elements and the method may further include disposing a resilient member between the first and second securing elements. As discussed previously, while the resilient member may serve to limit lateral movement of the portions of the tubular members, the first and second securing members may serve to limit lateral movement of the resilient member and thus also the portions of the tubular members. As another example, the first and second securing elements may be located on opposite sides of a longitudinal axis of at least one of the cover member and the sheet member.

In another embodiment, the cover member may be pivotally interconnected to the base member at an adjoinment region. The adjoinment region may include any devices or substances (e.g., stitching, sticky tape, adhesives, portion of the cover and base members) operable to allow the cover member to pivot or otherwise move relative to the base member. In this embodiment, the method may include arranging a free end of the tubular members to be near the adjoinment region. Such positioning of the free ends of the tubular members may enhance stabilization of the portions of the tubular members by limiting movement thereof relative to the cover and sheet members. In other embodiments, one or more absorbent members (e.g., drain sponges) may be disposed between the body site and the base member. For instance, the absorbent member may be placed over the body site after the body site has been appropriately cleaned, and then the base member may be placed over the body site.

In some embodiments, the method may include placing any appropriate garment over the cover member. It will be appreciated that the garment may serve to stabilize the base and cover members (and thus the portions of the tubular members) relative to the body site. In other words, the garment may serve to limit movement of the base and cover members and tubular members relative to the body site. As such, the garment need only be of a size and material that can urge the cover and base members against the body site at least to some degree. For instance, the garment may include an undergarment (e.g., bra, spandex). In one variation, the cover and base members may be inserted into any appropriate pocket or sleeve formed on the inside of the garment to further stabilize the cover, base and tubular members relative to the garment. For example, in the case of the base and cover members being in the form of first and second resilient members, the first and second resilient members may be inserted into the pocket to stabilize (e.g., at least partially immobilize) the first and second resilient members (and tubular member portions contained therewithin) relative to the garment.

In other embodiments, the method may include moving the cover member away from the base member and the tubular members. This step may advantageously allow the patient or personnel to adjust the tubular members, add or replace a resilient member, and the like. In one variation, the method may even include separating the tubular members from the base member. This step may be performed before each treatment session or also at the end of the entire treatment period. For instance, a radiation therapy procedure may be performed after the base member is separated from the tubular members. Sometime after separation, the method may further include second locating the portion of the tubular members through and over another base member, and second positioning another cover member over the another base member and the portions of the tubular members such that the portions of the tubular members are located between the another base and cover members. In this regard, another (e.g., new, sterile) base and cover member may be utilized after each time the old previous base member is separated from the tubular members (e.g., after each therapy session). In other embodiments, the same cover and base members may be used through and/or between multiple treatment sessions.

### BRIEF DESCRIPTION OF THE DRAWINGS

FIG. 1A illustrates a perspective view of an exemplary prior art brachytherapy apparatus in a collapsed configuration.

FIG. 1B illustrates a perspective view of the exemplary prior art brachytherapy apparatus of FIG. 1A in an expanded configuration.

FIG. 2 illustrates a perspective view of another exemplary prior art brachytherapy apparatus with a balloon in an expanded configuration.

FIG. 3 illustrates a perspective view of a stabilization system according to one embodiment.

FIG. 4 illustrates a perspective view of a percutaneously positioned brachytherapy apparatus and a drain sponge that may be usable as part of the stabilization system of FIG. 3.

FIG. 5 illustrates the perspective view of FIG. 4 but with the stabilization system being positioned over the drain sponge in an open configuration and anchored to the tubular members.

FIG. 6 illustrates the perspective view of FIG. 3 but with the stabilization system in a closed configuration.

FIG. 7 illustrates a perspective view similar to FIG. 5 but with a number of resilient members stacked to stabilize a rigid tubular member protruding away from the body site.

FIG. 8 illustrates a perspective view of a stabilization system according to another embodiment.

FIG. 9 illustrates a perspective view of a stabilization system according to another embodiment.

FIG. 10 illustrates a block diagram of one method of use of the stabilization systems disclosed herein.

FIG. 11 illustrates a perspective view of a garment including a pocket sized for receipt of any of the stabilization devices of FIGS. 3-9.

### DETAILED DESCRIPTION

Disclosed herein is a stabilization system and/or apparatus usable in conjunction with one or more brachytherapy devices used during a brachytherapy procedure. As will be more fully described below, the stabilization system may serve to stabilize one or more portions of a brachytherapy device that has been percutaneously positioned at a body site of a patient. For purposes of establishing context, two exemplary prior art brachytherapy devices will be initially discussed following by a discussion of the stabilization system.

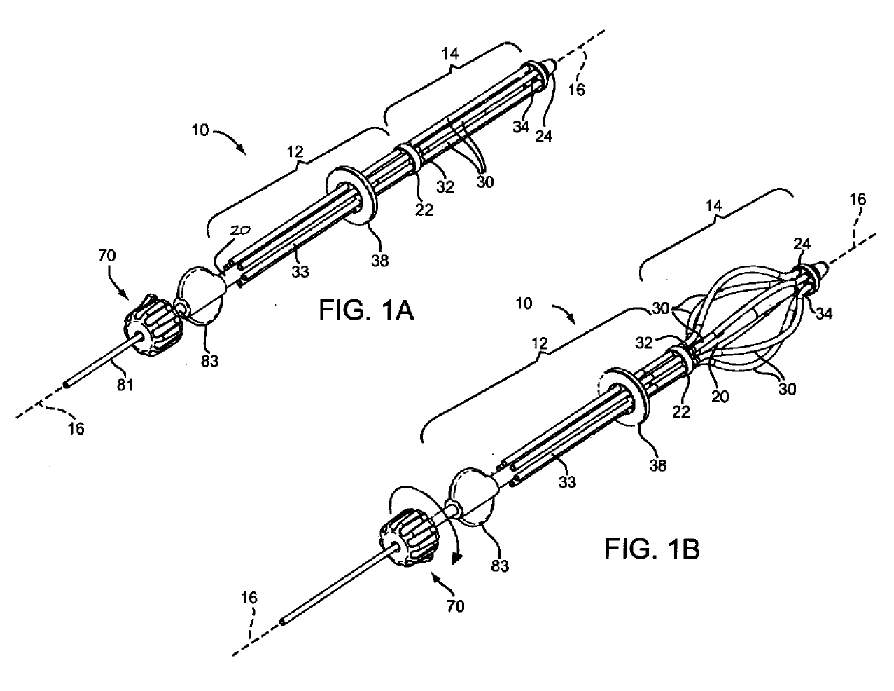

In this regard, FIGS. 1A and 1B illustrate an exemplary prior art expandable brachytherapy apparatus 10 for use during a brachytherapy procedure. The apparatus 10 may include a proximal or tail portion 12 and a distal or therapy delivery portion 14. A longitudinal axis 16 may extend between the proximal and distal portions 12, 14. The distal portion 14 may be deployed within a target location of a patient's body, e.g., a tumor or cavity within a breast or other body structure (not shown). The proximal portion 12 may extend from the distal portion 14, e.g., such that the proximal portion 12 protrudes at least partially outside of the body structure. The distal portion 14 may be movable between a collapsed configuration (as shown in FIG. 1A) for introduction through a tissue tract to a target location and a fully deployed or expanded configuration (as shown in FIG. 1B) for providing a three dimensional array of pathways or lumens at the target location.

The apparatus 10 may include an expansion tool 70 which may be coupled to the apparatus 10 for expanding and/or collapsing the distal portion 14. The expansion tool 70 may be detachable from or permanently attached to the apparatus 10. The apparatus 10 may also include one or more other components (e.g., sheath, cover) which may overly at least the distal portion 14 until deployment. In addition or alternatively, the apparatus 10 may be part of a system including a tubular delivery device (e.g., catheter, cannula, trocar, obturator, needle) for introducing the apparatus 10 into a target location. The apparatus 10 may also include a sharpened distal tip (not shown) to facilitate advancement directly through tissue.

The apparatus 10 may include an elongate core member 20 extending between a proximal hub 22 and a distal hub 24 and within a plurality of flexible elongate members 30 disposed around the core member 20 and/or extending between the proximal and distal hubs 22, 24. The core member 20 may be a substantially rigid member extending between the proximal and distal hubs 22, 24 but compressible and/or extendable axially to direct the proximal and distal hubs 22, 24 towards and/or away from one another and correspondingly cause the elongate members 30 to expand or collapse. For instance, the core member 20 may include first and second telescoping members (not shown) that may be appropriately interconnected (e.g., threaded connection) to cause the core member 20 to be compressible and/or extendable.

The elongate members 30 may be elongate, fixed length tubular members or “catheters,” each including a proximal end 32, a distal end 34, and a lumen (not shown) extending therebetween. The proximal ends 32 may be appropriately received in, through, and/or coupled to the proximal hub 22. Tubular extensions 33 may also be received in and/or coupled to the proximal hub 22 and/or coupled directly to the proximal ends 32 of the elongate members 30 (e.g., extending proximally from the proximal hub 22 to at least partially define the proximal portion 12 of the apparatus 10). Each tubular extension 33 may include an opening 33 that provides access into a respective lumen e.g., through the tubular extension 33 and into a respective elongate member 30) for receiving any appropriate radiation source. Alternatively, the tubular extensions 33 may be formed as an integral part of the elongate members 30 (e.g., as a continuous extrusion or molding) such that the elongate members 30 extend from the openings 33 to the distal ends 34.

The tubular extensions 33 may remain substantially free relative to one another or may be at least partially constrained relative to one another. For example, a collar 38 may be provided that includes openings for receiving respective tubular extensions 33 therethrough to thereby keep the tubular extensions 33 together, organized, and/or otherwise limit relative movement of the tubular extensions 33. The collar 38 may be fixed axially or may be movable axially relative to the tubular extensions 33. Moreover, one or more of the various components (e.g., tubular extensions 33) may be flexible.

To facilitate expansion and collapse of the apparatus 10, the expansion tool 70 may be appropriately coupled to a proximal end (not labeled) of the core member 20. For example, the proximal end of the core member 20 may include external threads, a hex head, or other connector (not shown), which may be used to connect the expansion tool 70 to the apparatus 10. In any event, the expansion tool 70 may be rotated or otherwise twisted to cause the expansion and collapse of the elongate members 30. A handle 83 may optionally be included on the core member 20 to facilitate connection and or use of the expansion tool 70. It will be appreciated that the expansion tool 70 may be removed after the elongate members 30 are expanded to allow for one or more treatment sessions. When it is desired to remove the apparatus 10, the expansion tool 70 may be reconnected to the apparatus 10 and used to collapse the elongate members 30, whereupon the apparatus 10 may be removed from the patient's body.

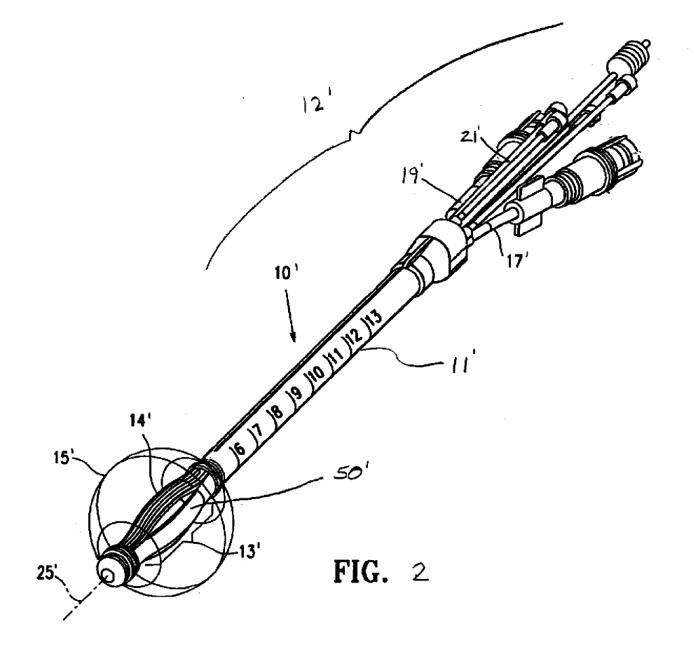

With reference to FIG. 2, another exemplary prior art expandable brachytherapy apparatus 10′ for use during a brachytherapy procedure is illustrated. The apparatus 10′ generally extends along a longitudinal axis 25′ and may include an elongated tube or shaft 11′ (which may be rigid), a proximal or tail portion 12′ and a distal or therapy delivery portion 14′ including a treatment portion 13′ that may be deployed within a target location of a patient's body for treatment. The apparatus 10′ may include a plurality of outer delivery tubes 21′ for administration of one or more radioactive members or substances into the target area or cavity of a patient. Each outer delivery tube 21′ is interconnected to a lumen (not shown) running through the elongated shaft 11′, and each lumen is interconnected to an inner delivery tube 50′ in the treatment portion 13′. The apparatus 10′ may also include a vacuum line 19′ for vacuuming out air pockets in the cavity and other appropriate componentry. The device 10′ also includes a balloon 15′ on the distal portion 14′ which surrounds the treatment portion 13′ and may be appropriately inflated within the cavity of the target area via an inflation line 17′. It will be appreciated that the apparatus 10′ may also include other components and features that will not be described in further detail.

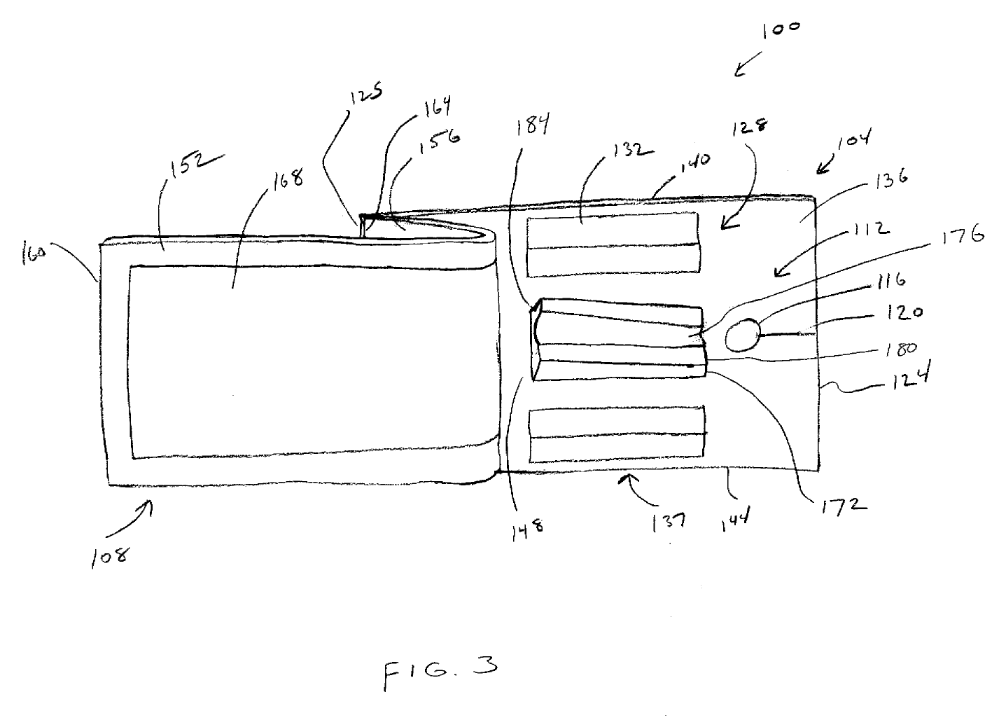

Referring now to FIG. 3, one embodiment of a stabilization system including a stabilization device 100 usable with a brachytherapy apparatus (e.g., the apparatuses 10, 10′ of FIGS. 1-2) is illustrated that may serve to stabilize one or more portions of the brachytherapy apparatus between treatment sessions. While the device 100 will be discussed in the context of a brachytherapy procedure, the stabilization device may be used in other medical contexts as well. The illustrated device 100 may include a base member in the form of a sheet member 104 that is positionable over a body site of a patient (e.g., adjacent to the location that the brachytherapy apparatus enters the patient's body, not shown in FIG. 3), and a cover member 108 that is positionable over the sheet member. Together, the sheet member 104 and cover member 108 may serve to stabilize one or more tubular members (e.g., tubular extension 33 from FIG. 1A, not shown in FIG. 3) or other components (e.g., wiring) associated with a brachytherapy apparatus that are located between the sheet and cover members 104, 108. In other arrangements or embodiments, the base and cover members may be in the form of first and second resilient members (e.g., foam) that may collectively receive or sandwich the one or more tubular members. As will be discussed in more detail in later sections, the first and second resilient members may be part of a common resilient member that has been sliced, cut or other formed to create a receiving area between the first and second resilient members for receipt of the one or more tubular members. Alternatively, the first and second resilient members may be separate pieces. In any case, the “one or more tubular members” will hereinafter be referred to as “tubular members”, although use of “tubular members” includes both a single tubular member and a plurality of tubular members.

The sheet member 104 may broadly serve to anchor the device 100 to a body site as well as stabilizing tubular members as will be described below. As shown, the sheet member 104 may be of any shape that allows the sheet member 104 to conform to or otherwise be draped over the body site and receive tubular members. For instance, the sheet member 104 may be of a rectangular shape, and/or may have a length dimension of 11 inches plus or minus 1 inch and a width dimension of 6 inches plus or minus 1 inch. It has been found that such dimensions adequately provide stabilization for one or more tubular members. In some arrangements, the sheet member may accommodate tubular members having a length (e.g., from the body site to a free end) of up to 9 inches or even greater. The sheet member 104 may also be in the form of one or more other shapes such as square-shaped, circular, triangular, and the like with various dimensions.

The sheet member 104 may be manufactured from any appropriate flexible material that may be durable. In one embodiment, the sheet member 104 may be composed of multiple layers (e.g., three layers) such as layers of spunbond polypropylene, polyethylene and Sontara® manufactured by DuPont™ of Wilmington, Del. In other embodiments, the sheet member 104 may be constructed of any appropriate breathable material such as a hydrophobic, breathable material that allows for passage of air while limiting the passage of liquids. In further embodiments, the sheet member 104 may include any appropriate non-allergenic materials. In further embodiments, the sheet member 104 may be constructed of a non-woven fabric. For instance, the sheet member 104 may be constructed of a spunbond/meltblown/spunbond (SMS) non-woven fabric that may include polymers such as polypropylene and/or polyethylene. SMS materials may provide advantageous features such bacteria impermeability, fluid penetration resistance, softness, comfort, and the like. In some embodiments, the sheet member 104 may be constructed of other types of materials that provide liquid and/or micro-organism repellency. In other embodiments, the sheet member 104 may also include a non-slip or non-skid surface or material to prevent or otherwise reduce undesired slipping or movement of the drape portion during a surgical/interventional procedure. Even further embodiments contemplate that the sheet member 104 may be constructed of a resilient member (e.g., foam) and/or contain fragrance or aromatherapy types of products such as oils or lotions to mask unpleasant odors in addition to improving the psychological and/or physical well-being of the patient. In additional embodiments, the sheet member 104 may include folds or creases (not shown) situated or disposed across portions thereof to assist a user in packaging the device 100.

The sheet member 104 may include one or more anchoring elements 112 that may be operable to anchor the sheet member 104 and thus the device 100 as a whole to tubular members emanating from a body site. As shown, the anchoring element 112 may be an opening in the form of a receiving aperture 116 and/or an access notch or slit 120. The receiving aperture 116 may be of any shape or size (e.g., circular, elongated slot) that is operable to receive one or more tubular members. For instance, the receiving aperture 116 may be a circular hole extending through the sheet member 104 with a diameter of ½ inch+/−0.125 inches. In another arrangement, the receiving aperture 116 may be elongated with dimensions of 1½ inches+/−0.25 inches by ¾ inch+/−0.125 inches.

The access slit 120 may allow a doctor or other personnel to locate the tubular members within the receiving aperture 116. Specifically, the access slit 120 may extend from the receiving aperture 116 to a first edge or end 124 of the sheet member and may allow personnel to push or otherwise position the sheet member 104 along the body site such that the tubular members travel through the access notch 120 and into the receiving aperture 116. Once the tubular members are received within the receiving aperture 116, the sheet member 104 and thus the device 100 may be temporarily anchored to the tubular members until the patient or personnel pulls on the sheet member 104 or device 100 or otherwise dislodges the tubular members from the receiving aperture 116. Other anchoring devices 112 are also contemplated such as press-fit devices, hook and loop arrangements, loops, and the like, and the anchoring element 112 is not limited by the receiving aperture 116 and access notch 120 disclosed herein. In some variations such as in the situation where multiple catheters or tubes are inserted into the body site through different openings in the body, use of anchoring elements 112 may not be needed, or the anchoring elements may be specifically modified to accommodate tubes emanating from the body at different locations (e.g., multiple holes and slits in the sheet member, multiple hook and loop straps).

With continued reference to FIG. 3, the device 100 may include one or more orientation or securing elements 128 each of which may be operable to at least removably secure the cover member 108 to the sheet member 104 and thereby secure and/or orient a portion of each of the tubular members between the sheet member 104 and the cover member 108. As will be discussed in more detail below, the portions of the tubular members may be secured between the cover and sheet members 108, 104 between radiation treatment sessions. Each securing element 128 may be in the form of an adhesive strip 132 (e.g., sticky tape) disposed over a top surface 136 of the sheet member 104 and may include a removable backing sheet (not shown) that may serve to protect the adhesive strip 132 or otherwise limit the adhesive strip 132 from adhering to a surface until the backing sheet is removed.

As shown, the sheet member 104 may include a plurality of adhesive strips 132, some of which may be generally adjacent a first side 140 of the sheet member 104 and some of which may be generally adjacent a second side 144 of the sheet member 104. In one embodiment, each adhesive strip may be about 1 inch wide by 5 inches long, and may be spaced about 2¼ inches from a second end 125 of the sheet member 104.

Furthermore, a receiving area 148 may be defined on a portion of the top surface 136 for receiving or otherwise accepting a portion of one or more tubular members. The receiving area 148 may be located between the adhesive strips 132 located adjacent the first and second sides 140, 144 such that in use, the interaction between the adhesive strips 132 and the cover member 108 may serve to restrain one or more tubular members in the receiving area 148 from lateral movement as well as upward or downward movement.

In one arrangement, the width of the receiving area 148 (e.g., distance between the adhesive strips) may be 1¾ inches+/−0.5 inches although other dimensions are also contemplated. It should be appreciated that the use of multiple adhesive strips 132 as shown in FIG. 3 may accommodate the increased height of resilient members 172 (which may be stacked) and/or bundles of tubular members.

More specifically, as this increased height may serve to pull sides of the cover member 108 inward when the cover member 108 is positioned over the tubular members and the sheet member 104, the inner adhesive strips 132 (e.g., closer or closest to receiving area 148) serve to ensure that the cover member 108 is removable secured to the sheet member 104. The resilient members 172 will be discussed more fully below.

Other arrangements are also envisioned such as one or more securing members 128 being located on other portions of the sheet member 104 as well as one or more portions of the cover member 108 (e.g., a bottom surface 152 of the cover member 104). Additionally, the one or more securing members 128 are not limited to adhesive strips; other arrangements such as hook and loop, snaps, ties, rubber bands, string, tape, glue and the like are also contemplated. For instance, instead of or in addition to adhesive strips 132, a technician may, after positioning the tubular members within the receiving area 148 and positioning the cover member 108 over the sheet member 104, secure one or more rubber bands around an outer surface of the sheet and cover members 104, 108.

Turning now to the cover member 108, the cover member 108 may broadly serve to stabilize tubular members received between the sheet and cover members 104, 108 and/or absorb or at least contain fluids or other debris generated as a result of a brachytherapy procedure. The cover member 108 may include top and bottom surfaces 156, 152 in addition to first and second ends 160, 164. In one variation, a portion of the bottom surface 152 may include an absorbent region 168 operable to generally cover and/or contact the tubular members when the cover member 108 is removably secured to the sheet member 104. In this regard, the cover member 108 may be operable to wick up or absorb fluids associated with the tubular members or other componentry. The cover member 108 may be of any appropriate dimensions and of any appropriate flexible and durable material(s). In one embodiment, the cover member 108 may have a length dimension of 11 inches plus or minus 1 inch and a width dimension of 5 inches plus or minus 1 inch and may be constructed of equal proportions of rayon viscose and polyester. In some embodiments, the cover member 108 may include any appropriate non-allergenic materials and/or resilient members (e.g., foam pieces).

Although not labeled, the cover member 108 may include a non-absorbent sheath or layer surrounding at least a portion of the absorbent region 168 which may serve to provide protection for the patient and personnel from fluid contact. For instance, the non-absorbent layer may extend across an entire surface of the top surface 156 and a portion of the bottom surface 152 (e.g., ¼ inch inward from a perimeter).

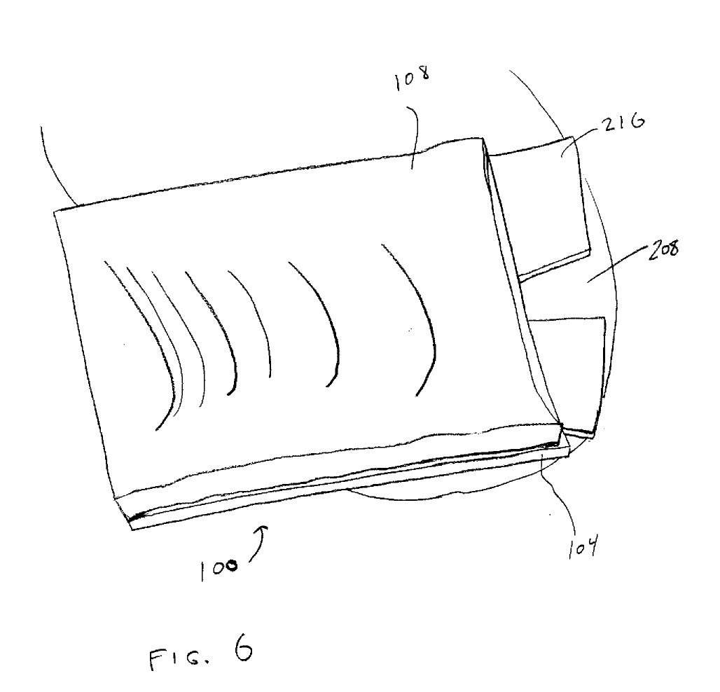

In some arrangements, the cover member 108 may be interconnected to the sheet member 104. For instance, the second end 125 of the sheet member may be interconnected to the second end 164 of the cover member 108 in any appropriate manner such as by adhesives, tape, stitching, etc. In this regard, the cover member 108 may be operable to pivot or otherwise move between an open configuration (e.g., as shown in FIG. 3) as a closed configuration (e.g., as shown in FIG. 6) such that the cover member 108 may be positioned over the sheet member 104.

As also seen in FIG. 3, the device 100 may include a resilient orientation member 172 in the form of a block of any appropriate material (e.g., foam) that may be removably located in the receiving area 148. Broadly, the resilient member 172 may provide additional stabilization of the tubular members received in the receiving area 148 and enhance patient comfort between radiation treatment sessions. Moreover, the resilient member 172 may serve to orient the tubular members between the sheet and cover members 104, 108 and over and/or within the receiving area 148. As the resilient member 172 may give or otherwise deform when tubular members are located thereon and the cover member 108 is removably secured over the sheet member 104, the resilient member 172 may serve to additionally limit lateral movement of the tubular members received thereon.

To enhance positioning of the tubular members on the resilient member 172, a groove or channel 176 may optionally be located on a portion of the resilient member 172 for receiving the one or more tubular members. The resilient member may include first and second ends 180, 184 and in one embodiment, the first end 180 may have a height less than that of the second end 184. In this regard, the resilient member may resemble a wedge member which can be placed in any desired orientation (e.g., the first end 180 being located adjacent or towards the anchoring element 112 or body site and the second end 184 be located away from the anchoring element 112 or body site or vice versa) to facilitate location of the tubular member in the receiving area 148.

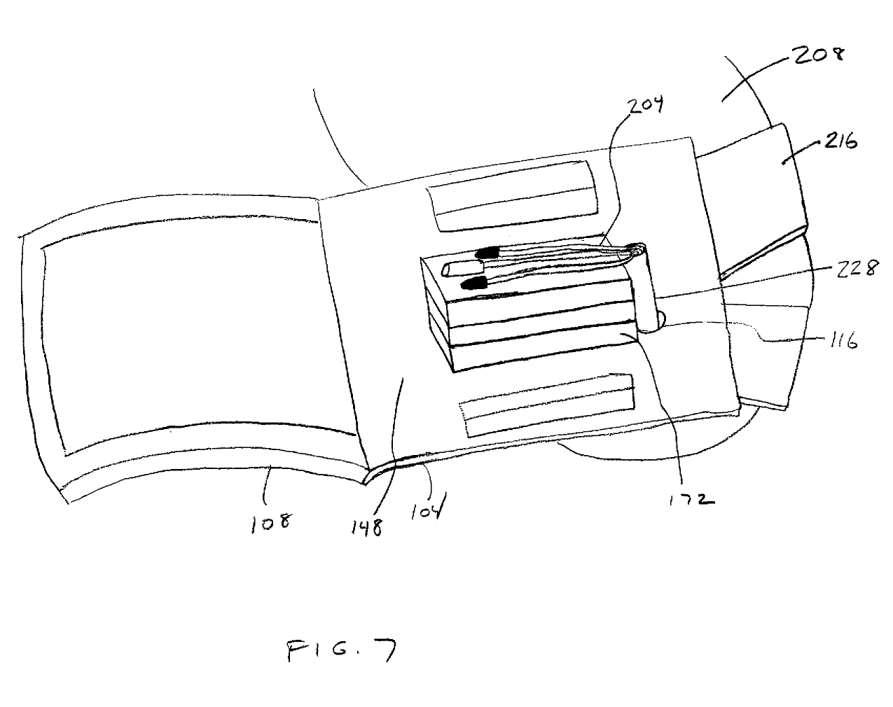

As an additional benefit, the resilient member 172 may provide a cushioning effect between the patient's body and the tubular members to limit the tubular members from irritating the patient and thus enhance patient comfort during the therapy period and between therapy sessions. Furthermore, additional resilient members 172 may also be included to provide additional levels of cushioning and/or enhanced stabilization of the tubular members. For instance, as some tubular members exiting the anchoring device 112 in a direction away from the body site may include relatively rigid members that resist deformation, one or more resilient members 172 may be stacked to provide additional stabilization of the rigid members. Stacking of resilient members 172 will be discussed in more detail below with respect to FIG. 7.

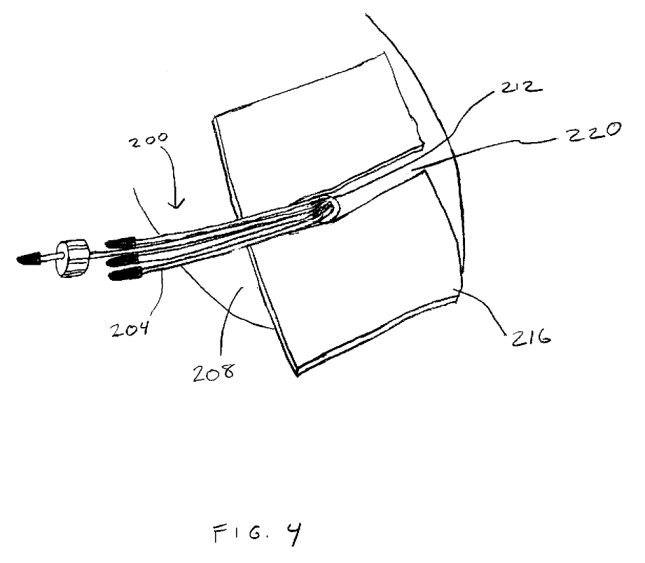

Referring now to FIGS. 4-6, the device 100 will be illustrated in conjunction with a brachytherapy procedure on a patient. Specifically, FIG. 4 illustrates a perspective view of any appropriate body site 208 (e.g., a breast) including a brachytherapy device 200 inserted therein and a sterile drain sponge 216 that may be usable with the device 100 positioned thereover. A representative brachytherapy procedure may utilize any appropriate brachytherapy device 200 (e.g., device 10 or 10′ of FIGS. 1A, 1B, 2) including one or more flexible tubular members 204 (e.g., tubular extensions 33 of FIG. 1A, 1B, elongated shaft 11′ and/or delivery tubes 21′ of FIG. 2). As illustrated, the tubular members 204 may extend from an area above and outside the body site 208 to an area within the patient's body such as a cavity within which a lumpectomy was performed (e.g., a “target site”) via a passageway 212 that extends from the body site to the target site. In this regard, the tubular members may be percutaneously positionable at the body site 208. It should be appreciated that some tubular member 204 may not necessarily extend from the portion outside the body site all the way to the target site. For instance, at least one tubular member 204 may extend partially into the passageway 212 whereby the tubular member 204 may be interconnected (e.g., fluidly interconnected or interconnectable) to another tubular member or component before reaching the target site.

In any case, before the device 100 is removably anchored to tubular members 204, one or more sterile drain sponges 216 (e.g., absorbent members) may be appropriately placed over the body site 208 and around the tubular members 204. While one or more drain sponges 216 will be discussed as being usable with the device 100, it should be appreciated that the one or more drain sponges 216 need not be used if, for instance, a bottom surface 137 of the sheet member 104 (e.g., the surface in contact with the body site of the patient) is itself already sterile. Each drain sponge 216 may be operable to provide a sterile layer and/or barrier between the body site 208 and the device 100 and absorb or otherwise collect fluid or debris associated with the brachytherapy procedure. As shown, the drain sponge 216 may include an anchoring element 220 such as but not limited to a slit, slot or passageway that may allow the drain sponge 216 to be wrapped or placed around the tubular members 204 and thus temporarily anchored to the tubular members 204. In some embodiments, the anchoring element may additionally include a receiving aperture and in this regard may be similar to the anchoring element 112 of FIG. 3.

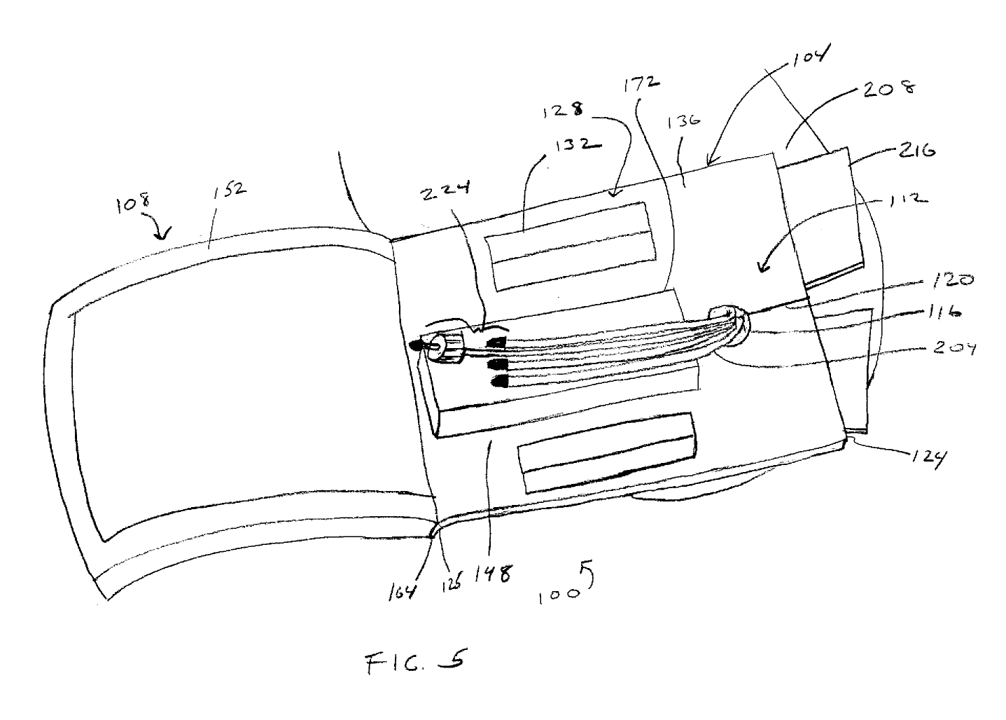

Turning now to FIG. 5, the perspective view of FIG. 4 is illustrated but with the device 100 being positioned over the drain sponge 216 in an open configuration and anchored to the tubular members 200. The device 100 has been anchored to the tubular members 204 by way of the anchoring element 112 (e.g., positioning the tubular members 204 through the access notch 120 and into the receiving aperture 120) and the tubular members 204 have been positioned over the resilient member 172 (channel 176 and wedge shape not shown) and within the receiving area 148. In one arrangement, ends 224 of the tubular members 204 may be positioned at least generally adjacent to an intersection (e.g., pivotal interconnection) of the sheet member 104 and the cover member 108. Such positioning of the ends 224 enhances stabilization of the tubular members 204 because of the decreased distance between the top surface 136 of the sheet member 104 and the bottom surface 152 of the cover member 108. That is, by urging the ends 224 of the tubular members 204 towards the intersection, the ends 224 may become at least partially “wedged” between the top surface 136 of the sheet member 104 and the bottom surface 152 of the cover member 108 (e.g., when the device is in the closed configuration) which may limit lateral as well as upward and downward movement of the tubular members 204.

After the tubular members 204 have been appropriately positioned in the receiving area 148 and/or over the resilient member 172, any backing sheets on the adhesive strips 132 may be removed and the cover member 108 may be pivoted or otherwise moved or positioned into a closed configuration such that the cover member 108 is removably secured to the sheet member 104 via the securing elements 128 and thereby covers and stabilizes the tubular members 204. It should be noted though that the backing sheets may also be removed before the tubular members 204 have been positioned in the receiving area 148. With reference to FIG. 6, the cover and sheet members 108, 104 at least substantially cover and shield the tubular members 204 (not shown in FIG. 6) which may provide a number of benefits and advantages to the patient, medical personnel, and the like.

More specifically, the device 100 may serve to stabilize or otherwise limit movement of the tubular members 204 relative to the sheet and cover members while limiting access to the tubular members 204 because the tubular members 204 may be captured between the sheet and cover members 104, 108. Additionally, when a garment (e.g., bra) is placed over the device 100, the tubular members may also be stabilized relative to the body site. In this regard and as will be described more fully below, movement of the “treatment portion” of the brachytherapy device within the target area or cavity of the patient may be reduced.

Turning now to FIG. 7, the device 100 is illustrated in a manner that may accommodate one or more rigid tubular members extending or otherwise protruding from the body site 208 and in this regard, may be useful with the brachytherapy device 10′ of FIG. 2. Here, a rigid (e.g., at least substantially non-bendable) tubular member 228 (e.g., elongated shaft 11′ of FIG. 2) may extend or otherwise protrude from the body site and through the receiving aperture 116, and one or more flexible tubular members 204 (e.g., delivery tubes 21′ of FIG. 2) may extend or protrude from the rigid tubular member 228. As it may be difficult to bend or otherwise position the rigid tubular member 228 into the receiving area 148, the inventors have discovered that a number of resilient members 172 may be built or stacked up to enhance stabilization (e.g., limit side to side movement) of the rigid tubular member 228 and/or reduce kinking and/or strain of the tubular members 204.

For instance, by providing a resilient member 172 that has a height close to or greater than a height of the rigid tubular member 228 or else stacking resilient members 172 to reach such a height, kinking and strain of the tubular members 204 adjacent to where the tubular members 204 exit the rigid tubular member 228 (so as to extend into the receiving area 148) may be limited or otherwise reduced which may reduce wear of the brachytherapy device in addition to enhancing stabilization of the tubular members 204. That is, greater contact may be achieved between the cover member 108 and the uppermost resilient member 172 compared to the situation in which upon exiting the rigid tubular member 228, the tubular members 204 were bent or kinked substantially downward in a direction towards the sheet member 104. In one arrangement, the combination of the sheet and cover members and resilient member(s) may accommodate exit angles (e.g., the angle between the body site and the tubular member at the location that the tubular member exits the body site) of up to 40° and greater.

Other orientations, shapes and sizes of one or more resilient members 172 to provide appropriate positioning and stabilization of the various componentry of a brachytherapy device (e.g., tubular members) are also envisioned as being within the scope of the embodiments. For instance, while the resilient members 172 have been shown in block form without receiving channels and not being in a wedge form, in some embodiments one or more resilient members 172 each having a receiving channel and/or being in a wedge form may be utilized to achieve a desired orientation of the tubular members.

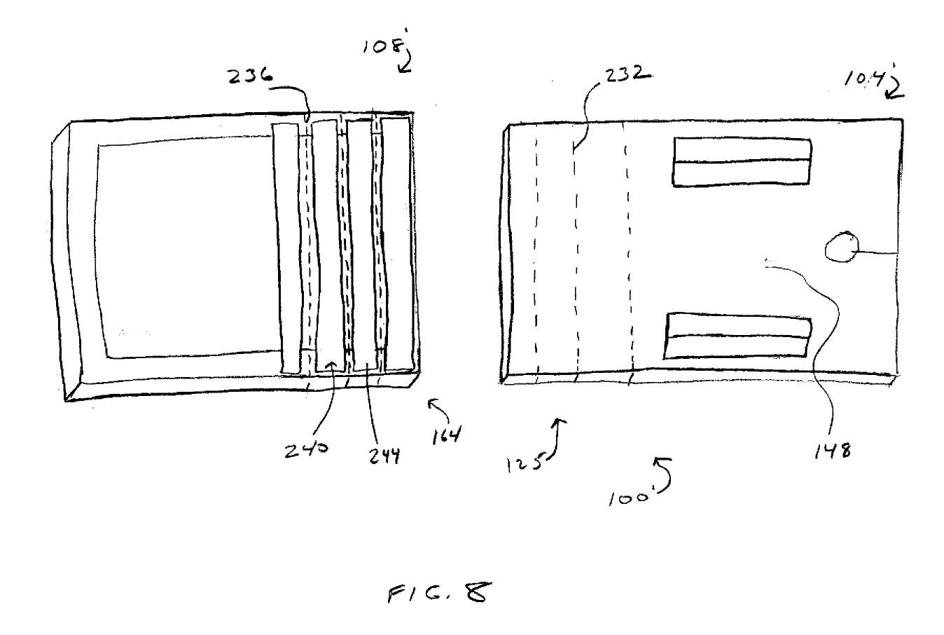

In some embodiments, one or more components of the stabilization device 100 may be appropriately adjustable in size and/or shape to accommodate patients and brachytherapy devices of various sizes and configurations. With reference now to FIG. 8, another embodiment of a stabilization device 100′ is illustrated that includes sheet and cover members 104′, 108′ each of which may be adjustable in size and/or shape. The sheet and cover members 104′, 108′ may initially be separated (e.g., not interconnected) to allow each to be individually adjusted. For instance, the sheet member 104′ may include one or more adjustability lines 232 (e.g., perforations, score lines, grooves, colored line) in any appropriate portion of the sheet member 104′ and in any appropriate shape. As shown, the sheet member 104′ includes a series of adjustability lines 232 each of which may extend at least partially through the sheet member 104′ and be in the form of a perforation extending between opposing edges of the sheet member 104′ (e.g., from one lateral side to the other lateral side).

Similarly, the cover member 108′ may also include a series of adjustability lines 236 (e.g., similar to adjustability lines 232). Additionally, the cover member 108′ (and/or sheet member 104′) may also include a number of securing elements 240 each of which may be in the form of an adhesive strip 244 (e.g., sticky tape) extending between the adjustability lines 232. In use, the patient or hospital personnel may appropriately adjust the sheet and/or cover member 104′, 108′ by way of separating one or more portions of the sheet and/or cover member 104′, 108′ along the adjustability lines 232, 236. For instance, the patient or technician may appropriately tear or cut (e.g., with scissors) the sheet and/or cover member 104′, 108′ along the adjustability lines to achieve a desired shape and/or size of the sheet and/or cover member 104′, 108′. Thereafter, the patient or technician may remove any backing strips associated with one or more of the adhesive strips 244 remaining on the cover member 108′ (e.g., the adhesive strip 244 located closest to the second end 164 of the cover member 108′), and then adhere the second end 164 of the cover member 108′ to the second end 125 of the sheet member 104′. At this point, the device 100′ may be utilized in a similar manner to the device 100 as discussed above. It is envisioned that the relative distance between the adjustability lines 232 and 236 may be similar such that if, for instance, two portions of the sheet member 104′ and two portions of the cover member 108′ are removed, the cover member 108′ may still be operable to appropriately cover and be positioned over the receiving area 148 (e.g., the sheet and cover members 104′, 108′ may have similar lengths).

It is contemplated that the device 100′ may be adjusted even after the cover member 108′ is secured to the sheet member 104′ along their ends. For instance, upon interconnecting the ends of the sheet and cover members 104′, 108′ and thereafter determining that one or more of the sheet and/or cover member 104′, 108′ needs adjusting, the patient or technician may separate the sheet and/or cover members 104′, 108′ along the adjustability lines 232, 236, remove any backing layers associated with the adhesive strip 244 located closest to the second end 164, and then adhere the cover member 108′ back onto the sheet member 104′. Additionally, while the securing elements 240 have been shown as adhesive strips 244, other forms are contemplated such as hook and loop elements (e.g., one hook and loop element secured to the sheet member 104′ and a corresponding hook and loop element secured to the cover member 108′), snaps, and the like. Although not shown, one or more of the resilient members 172 and drain sponges 216 may also be similarly adjusted (e.g., via separating one or more portions along perforation lines or with a tool such as scissors).

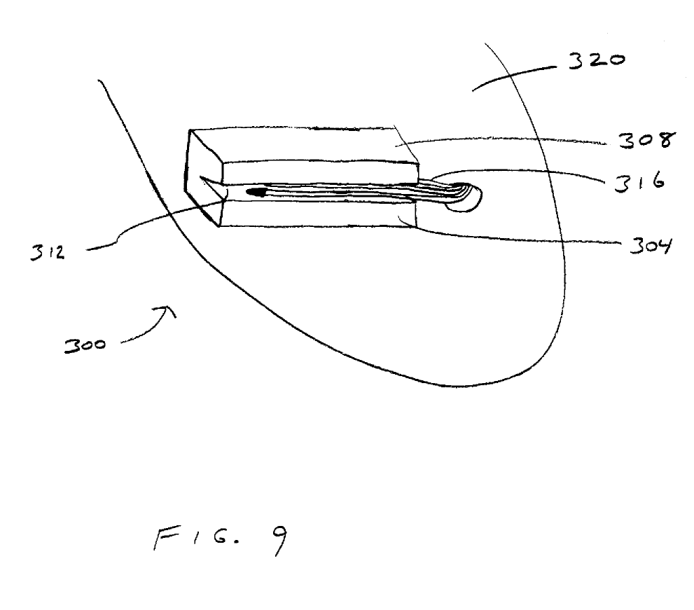

With reference now to FIG. 9, another embodiment of a stabilization device 300 is illustrated that includes base and cover members 304, 308 (e.g., first and second “clamshells”) along with a receiving area 312 therebetween. Portions of one or more tubular members 316 protruding from a body site 320 may be positioned within the receiving area 312 and thereafter sandwiched by the base and cover members 304, 308. The stabilization device 300 may be in the form of one or more resilient members that has or have been appropriately sliced, cut or otherwise formed (e.g., during a molding or extrusion process) along a length thereof to form the receiving area 312, base member 304 and cover member 308. For instance, each of the base and sheet members 304, 308 may be in the form of one or more pieces of foam and/or the above described resilient member 172.

In use, a technician or personnel may locate or otherwise position the portions of the tubular members 316 within the receiving area 312 and thus between the base and cover members 304, 308. Thereafter, the base and cover members 304, 308 may be appropriately removably secured together using one or more of the above discussed securing elements (e.g., adhesive tape, rubber band(s), hook and loop) to stabilize the portions of the tubular members relative to at least one of the base and cover members 304, 308. One or more portions of the stabilization device 300 may include one or more of the above discussed anchoring elements to removably secure or anchor the stabilization device 300 to the tubular members 316 and relative to the body site 320. Furthermore, one or more portions of the stabilization device 300 may include channels or other features (not shown, see previous discussion) to secure the portions of the tubular members against lateral and other movement. For instance, each of the base and cover members 304, 308 may include a channel on an inside surface thereof (e.g., that overlie each other) to create a substantially cylindrical channel for receipt of the tubular members.

It should be appreciated that, while the stabilization device 300 has been illustrated as being a single piece resilient component and/or a one-piece unit, it is envisioned that the stabilization device 300 may utilize individual components for the base and cover members 304, 308. Further, one or more of the stabilization devices disclosed herein may be used in conjunction. For instance, it is envisioned that the tubular members may be initially positioned between the base and cover members 304, 308 of the stabilization device 300, and then the stabilization device 300 may be positioned between the sheet and cover members 104, 108 of the stabilization device 100. Other arrangements are also contemplated.

In some embodiments, one or more kits may be arranged including one or more stabilization devices, drain sponges and resilient members. For instance, one kit may include a stabilization device, a drain sponge, and three resilient members of different shapes and sizes. For instance, the resilient members may include three wedge-shaped resilient members, the first being ¼ inch thick, the second being ¾ inches thick and the third being 1½ inches thick (at the highest end), whereby each is three inches long and two inches wide. In another example, two wedge-shaped resilient members may be included: the first being 1″ thick at one end, ½″ thick at the other end, 4″ long, 2″ wide, and having a groove that is 1″ wide and ¾″ deep, and the second being similar to the first but being 6″ long. It has been found that the above sizes and shapes of the resilient members advantageously accommodate the stabilization of tubular members of various shapes and sizes.

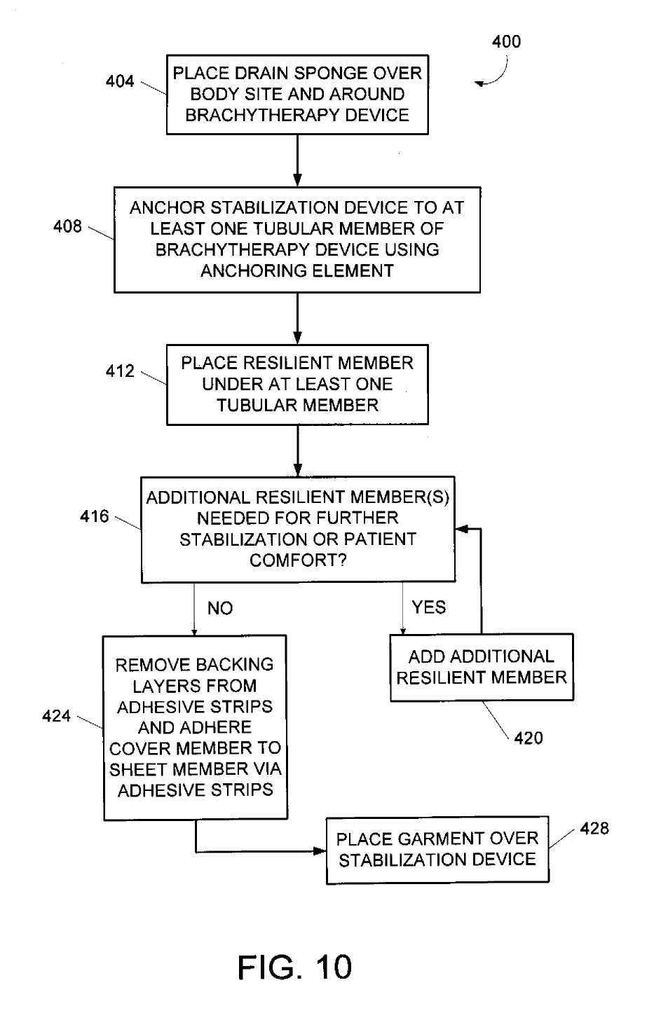

With reference now to FIG. 10, a flow diagram of one method 400 of using any of the stabilization devices disclosed herein is illustrated. It will be appreciated though that other methods of using the stabilization devices of the embodiments disclosed herein are also contemplated. It should also be appreciated that the method may be performed with hospital gloves on (e.g., latex gloves) due to the construction and design of the stabilization devices disclosed herein. In step 404, a drain sponge (e.g., see FIG. 3) may be placed over the body site of a patient undergoing a brachytherapy procedure and around the brachytherapy device. It will be appreciated for purposes of this method 400 that the brachytherapy device (e.g., see FIG. 1A, 1B or 2) has already been percutaneously positioned at a body site and that one or more tubular members emanates from and protrudes away from the body site. In this regard, the drain sponge may be positioned around one or more tubular members of the brachytherapy device (by inserting the tubular members into a slot in the drain sponge). It should be noted that the body site may be appropriately sterilized before positioning of the drain sponge.

In any event, a stabilization device (e.g., see FIGS. 3 and 5-8) may be anchored or otherwise removably secured to the tubular members of the brachytherapy device using one or more anchoring elements in step 408. For example, the tubular members may be inserted through an access channel and into a receiving aperture whereby the stabilization device becomes removably anchored to the tubular members. As previously discussed though, one or more other manners of anchoring the stabilization device are also envisioned as being within the scope of the embodiments disclosed herein.

In step 412, a resilient member (e.g., see FIGS. 3 and 5) may be placed under the tubular members and over the receiving area of the stabilization device to orient the tubular members within the receiving area of the stabilization device in addition to providing a cushioning effect for the patient. Step 416 then queries whether additional resilient members are needed for further stabilization, orientation and/or patient comfort. If the answer is yes, then one or more additional resilient members may be added in step 420. For instance, one or more resilient members of any appropriate shape (e.g., block-shaped, wedge-shaped) may be stacked over the first resilient member or otherwise appropriately positioned to achieve a desired positioning or stabilization of the tubular members or else a desired patient comfort level. When no addition resilient members are needed, then in step 424, the backing layers from the adhesive strips may be removed and the cover member of the stabilization device may be removably adhered to the sheet member via the adhesive strips. For instance, this step may involve pivoting or otherwise moving the cover member from an open to a closed configuration or position such that the cover member contacts and covers the tubular members to stabilize the tubular movements against movement relative to the stabilization device. It will be appreciated that other securing elements are also contemplated that might not have backing layers or sheets. Of course, the cover member may be opened at any time to readjust and/or add components (e.g., resilient members), and then closed again.

Once the cover member is secured to the sheet member and the tubular members are thereby secured and stabilized therebetween, any appropriate garment may be placed over the stabilization device in step 428. For instance, the patient or hospital personnel may position a bra over the stabilization device to removably secure the stabilization device to the patient and thus limit the stabilization device and tubular members captured therewithin from moving relative to the body site. In this regard, the “treatment portion” of the brachytherapy device (e.g., 14 or 14′ in FIG. 1A or 2) may be limited in the degree that it moves relative to the target site or cavity in the patient and thus costly imaging procedures (e.g., scout x-rays, CT scans) to confirm that the treatment portion has not moved between therapy sessions (e.g., when the patient has gone home at night in the middle of the 5-7 day treatment period) may be reduced.

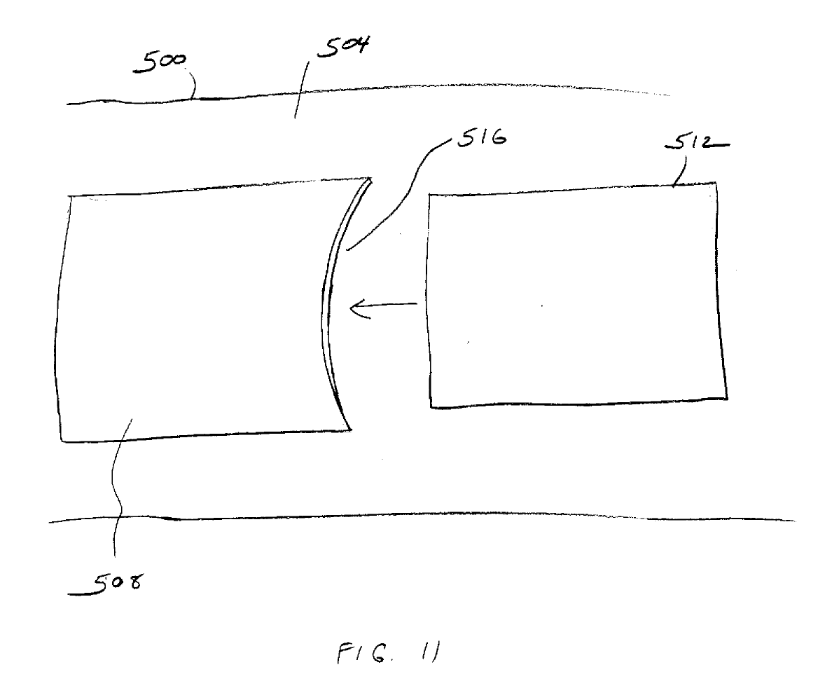

To enhance an interconnection between the garment and the stabilization device, any appropriate removable attachment or receiving device may be utilized. For instance, corresponding portions of hook and loop material may be attached to the stabilization device and garment and then removably interconnected when the garment is brought over the stabilization device. In another arrangement and with reference now to FIG. 11, a garment 500 may include one or more sleeves or pockets 508 appropriately attached to a surface thereof (e.g., inside surface 504) in any appropriate manner (e.g., via stitching or adhesives) that may be operable and/or sized to accommodate or otherwise receive a stabilization device 512 (e.g., any stabilization devices disclosed herein, tubular members not shown). In one embodiment, the pocket 508 may be an appropriately sized piece of fabric (e.g., spandex) and the like that has been secured to the inside surface 504 of the garment 500 around the entire perimeter thereof except for an entrance area 516 for the stabilization device 512. For instance, after appropriately stabilizing the tubular members relative to the stabilization device 512, the stabilization device 512 may be inserted into the pocket 508 on the garment 500 to removably secure the stabilization device 512 and tubular members relative to the garment 500 and also to the body site (not shown) once the garment 500 is positioned on the patient.

In another embodiment, one or more sides of the pocket 508 may be removably interconnected to the inside surface 504 of the garment 500 via any appropriate securing element(s) (e.g., hook and loop, adhesives, snaps, not shown). For instance, a patient or user may temporarily remove the one or more sides of the pocket 508 from the inside surface 504, place the stabilization device 512 (with tubular portions therewithin) adjacent or near a portion of the pocket 508, and then flap or otherwise position the pocket 508 over the stabilization device 512 and secure the pocket 512 to the inside surface 504. At this point, the stabilization device 512 is stabilized relative to the garment 500.

In a further arrangement, one or more portions of the garment 500 and/or stabilization device 512 may include any appropriate temperature control or adjustment arrangement (not shown). For instance, the garment 500 may include another pocket sized for receipt of a therapy pack (e.g., reusable hot/cold gel pack) that may be operable to limit and/or control temperature increase and/or decrease of the tubular members. The another pocket may be disposed beneath the pocket 508 such that the therapy pack may be received within the another pocket and over the inside surface 504 and the stabilization device 512 may be received within the pocket 508 and over the another pocket (and thus the therapy pack). This design allows the stabilization device 512 (and tubular members therewithin) to be in close relation to the therapy pack. Additionally or alternatively, one or more portions of the stabilization device 512 (e.g., cover member) may include pockets or other similar features sized for receipt of one or more therapy packs. In another variation, a portable sorption-based heat exchange device may be inserted into one or more of the pockets to initiate heat exchange (e.g., cooling) of the tubular members and/or other components and/or tissue.

Other removable attachment devices are also envisioned such as snaps, clips and the like which may be attached to the stabilization device and/or the garment. Moreover, any appropriate removable attachment device may also be utilized to enhance an interconnection between the stabilization device and the patient (e.g., the body site). For instance, one or more adhesive strips or other devices (e.g., tape, string) may be connected to a bottom surface of the sheet member. In this regard, any backing strips associated with the adhesive strips may be removed and the adhesive strips may be used to adhere the sheet member to the body site or other portion of the patient.

As discussed above, one or more portions of the stabilization device may be appropriately adjusted in one or more manners. Furthermore, it is envisioned that the stabilization device may remain on the patient until the following treatment session at which time the stabilization device may be appropriately removed (e.g., remove garment, lift cover, remove stabilization device from tubular members) and the treatment session would be performed. Thereafter, another (e.g., new) stabilization device may be anchored to the tubular members as described above until the following treatment session.

However, it is also contemplated that the same stabilization device may be utilized through multiple treatment sessions and/or for an entire treatment period. For instance, the stabilization device may remain at least partially interconnected to the tubular members and/or garment during one or more treatment sessions. More specifically, while the cover member may be removed from the sheet member, the sheet member may remain removably connected to the tubular members. Thus, the tubular members may be freely accessible by personnel to perform a therapy or treatment session while the stabilization device remains removably secured to the tubular members. In the case of the stabilization device 300 of FIG. 9 and upon initially securing the tubular members 316 to the stabilization device 300, the free ends of the tubular members 316 (e.g., where lumen access is provided) may be arranged to extend or protrude a portion past the stabilization device 300. As a result, the sheet member 304 may remain removably secured to the cover member 308 during a therapy session. In other embodiments, one or more portions of the stabilization may include various colors, designs and the like that may signify manufacturer or supplier logos, instructions, and the like.

Other steps are also contemplated. For instance, when it is desired to separate the stabilization device from the tubular members and thus the body site, this can be done with little or no movement of or damage to the tubular members such as tearing and/or pulling. Particularly, the technician or personnel may initially grasp the tubular members at a portion between the base member and the body site after the garment has been removed to expose the device. Thereafter, the technician may pull on the device with enough force to dislodge the tubular members from the opening and separate the device from the tubular members. The drain sponge may then be removed and the tubular members may be appropriately interconnected to a radiation source for a subsequent therapy session.

One or more of the components discussed herein (e.g., base member, cover member) may be constructed of any appropriate biodegradable or compostable material. For instance, one or more of the components may be constructed of a cornstarch-based biodegradable material. Other materials are also envisioned.

The above-noted embodiments are for the purpose of illustration and are not intended to limit the scope of the present invention or patent. Rather, various modifications, adaptations and extensions of the invention will be apparent to those skilled in the art and are intended to be within the scope of the present invention as contemplated by the claims that follow.

1-34. (canceled)

35. A method for use in connection with a radiation therapy procedure in which a radioactive substance is administered through at least one tube that is percutaneously positionable at a body site, the method comprising:
locating at least a portion of the at least one tube over a base member; and
positioning a cover member over at least a portion of the base member and the portion of the at least one tube, wherein the portion of the at least one tube is located between the cover member and the base member to stabilize the portion of the at least one tube in a desired position.

36. The method of claim 35, wherein the base member comprises a sheet member, wherein the locating step further comprises:
routing the portion of the at least one tube through an opening in the sheet member.

37. The method of claim 35, further comprising:
orienting the portion of the at least one tube relative to at least one of the base member and the cover member.

38. The method of claim 37, wherein the base member comprises a sheet member and wherein the orienting step further comprises:
disposing a resilient member between the portion of the at least one tube and the sheet member.

39. The method of claim 38, wherein the disposing step further comprises:
placing the resilient member along a longitudinal axis of the sheet member.

40. The method of claim 38, wherein the resilient member comprises a first end having a first height and a second end having a second height that is greater than the first height, wherein the disposing step further comprises:
arranging the first end to be near the body site; and
arranging the second end to be away from the body site.

41. The method of claim 38, wherein the resilient member comprises a channel in a surface thereof, wherein the method further comprises:
aligning the portion of the at least one tube with the channel.

42. The method of claim 38, wherein the resilient member comprises foam.

43. The method of claim 37, wherein the orienting step further comprises:
removably securing the cover member to the base member using at least one securing element.

44. The method of claim 43, wherein the at least one securing element comprises first and second securing elements that are connected to at least one of the cover member and base member, wherein the method further comprises:
disposing a resilient member between the first and second securing elements.

45. The method of claim 44, wherein the first and second securing elements are located on opposite sides of a longitudinal axis of at least one of the cover member and the base member.

46. The method of claim 35, wherein the cover member is pivotally interconnected to the base member at an adjoinment region, wherein the method further comprises:
arranging a free end of the at least one tube to be near the adjoinment region.

47. The method of claim 35, wherein the locating step further comprises:
disposing the portion of the at least one tube along a length of the base member.

48. The method of claim 35, further comprising:
disposing an absorbent member between the body site and the base member.

49. The method of claim 35, further comprising:
placing a garment over the cover member.

50. The method of claim 49, further comprising:
inserting the cover member and base member into a pocket associated with the garment.

51. The method of claim 49, wherein the garment comprises an undergarment.

52. The method of claim 51, wherein the undergarment comprises a bra.

53. The method of claim 35, further comprising:
moving the cover member away from the base member and the at least one tube.

54. The method of claim 53, further comprising:
separating the at least one tube from the base member.

55. The method of claim 54, further comprising:
performing a radiation therapy procedure.

56. The method of claim 54, further comprising:
second locating the portion of the at least one tube over another base member; and
second positioning another cover member over the another base member and the portion of the at least one tube, wherein the portion of the at least one tube is located between the another base and cover members.
